(19) United States
(12) Patent Application Publication
(10) Pub. No.: US 2016/0166726 A1
(43) Pub. Date: Jun. 16, 2016

(54) NON ADHERENT HEALING INTERFACE AND ASSOCIATED COMPRESS AND DRESSING

(71) Applicant: GERGONNE SAS, Oyonnax (FR)

(72) Inventors: Charles GERGONNE, Arbent (FR); Bertrand GERGONNE, Oyonnax (FR); Frédéric ROBÉ, Agencourt (FR)

(73) Assignee: GERGONNE SAS, Oyonnax (FR)

(21) Appl. No.: 14/966,313

(22) Filed: Dec. 11, 2015

(30) Foreign Application Priority Data
Dec. 11, 2014 (FR) 14/62248

(51) Int. Cl. A61L 15/60; A61L 15/50; A61L 15/28; A61L 15/58; A61L 15/18

(52) U.S. Cl. CPC A61L 15/60; A61L 15/58; A61L 15/18; A61L 15/28; A61L 15/50

(57) ABSTRACT

The present invention relates to a non adherent healing interface including at least one net with open meshes and wherein the net has been coated with a mixture including at least silicone gel and at least a gelling agent. Another object of the present invention is a compress and a dressing which includes at least the interface.

The present invention relates to a sterile non adherent healing interface intended to be applied directly in contact with a wound, as well as a compress and dressing comprising such an interface.

Among the medical devices, there are non adherent healing interfaces which are intended to be applied directly on a wound with a view to healing it, even accelerate the natural healing process.

Such interfaces are often designed to be applied on acute, chronic and sometimes exudative wounds of weakened people (for example people with severe burns, people at the end of life).

Thus, it is essential that the interfaces be designed for creating optimal conditions required for the healing process while being the least traumatic as possible for the patient, during the pose and especially the removal of the interface.

Non adherent healing interfaces known from the prior art are not fully satisfactory for the following reasons:

their placing and hold on the wound are not always easy as they do not always have suitable immediate adhesive properties,

their removal from the wound may be traumatic,

the regular and rapid healing of the wound is not always obtained, or very difficultly.

It is known that the healing of a wound can only evolve favorably if the interface does not adhere to the newly regenerated tissue and if the exudate is eliminated by creating a humid environment around and on the wound.

This is why, it is required to have an interface which is perfectly non adherent on the regenerated tissue, and which creates and maintains conditions of optimal humidity favorable to healing, while avoiding the risk of maceration.

The present invention perfectly fulfills these aforementioned objectives by proposing a new non adherent healing interface which includes at least one net with open meshes and which is characterized in that the net has been coated with a mixture comprising at least silicone gel and at least a gelling agent.

In a preferred manner, at least part of said meshes are not obstructed by said mixture comprising at least silicone gel and at least a gelling agent.

In one embodiment of the invention, most of said meshes are not obstructed by said mixture comprising at least silicone gel and a gelling agent.

In one embodiment of the invention, no mesh of the net is obstructed by said mixture.

The mixture with which the net of the interface is coated, substantially comprises silicone gel. Preferably, the weight in silicone gel represents at least 80%, preferably 80% to 95%, of the total weight of said mixture.

Silicone gel is known and suitable for temporarily making a medical device adhere to the skin. The silicone gel provides high adherence with the skin and a low liberating force by detachment. Preferably, the adhesive force of the silicone gel comprised in said mixture ranges between 0.5 N/cm and 2 N/cm.

Within the scope of the present invention, the silicone gel contained in the mixture coated on the net of the interface is in cross-linked form. Thereby, it has a certain cohesion.

Within the scope of the present invention, gelling agent, means a compound which forms a gel in aqueous medium, for example in water.

The advantage of the interface according to the invention is that it lightly adheres to the wound because the silicone gel of said mixture with which the net is coated is slightly adhesive. This property facilitates placing the interface on the wound of the patient and possibly, allows, replacing it if necessary.

Furthermore, because the net includes open meshes which are preferably mostly not obstructed by the silicone gel and gelling agent mixture, the wound exudates are gradually expelled as the healing takes its course, and while they pass through the meshes of the net, the gelling agent contained in said mixture will be hydrated and will form a gel. In other words, when crossing the net coated with a mixture substantially comprising silicone gel and at least one gelling agent, part of the exudates is captured by the hydrophilic nature of the gelling agent. The latter thus progressively transforms into a humid gel by:

thus creating an environment around the wound which is favorable to wound healing, even for wounds which are difficult to heal, such that the wound is partially or even totally healed.

making the interface absolutely non-traumatic during removal. Indeed, the gelling agent transformed into a gel tends to decrease the aforementioned adhesive properties of the silicone gel and thus makes the interface according to the invention absolutely non adherent; thus maintaining the integrity of the new scar tissue which is more fragile.

Thus, the association of the silicone gel and the gelling agent imparts the interface according to the invention with properties which are extremely favorable to healing:

the silicone gel provides good physical stability to the interface which may stay in place on the wound for several days, without moving or becoming dispersed and without adhering to the freshly regenerated tissue, while allowing bonding with a wide range of absorbent materials such as polyurethane foam, and thus as will be explained hereinafter,

the gelling agent which is gradually transformed into a gel as the exudates are evacuated allows maintaining a degree of humidity favorable to the healing process, while preventing the wound from drying out and, consequently, either forming a crust, or the interface adhering to the wound.

The non adherent healing interface according to the invention comprises a net with open meshes.

The net is advantageously formed from yarns which may be produced from a flexible material, hardly extensible or even non elastic. Said net may have been obtained by any weaving or knitting method which is perfectly within range of those skilled in the art.

The net may be perfectly stiff (in other words the meshes are blocked) or slightly extensible (with an extensibility advantageously ranging between 20% and 50%) in the warp direction and/or the weft direction.

In a preferred manner, the diameters of the yarns are the same in the warp and weft directions so as to allow an even coating of the mixture which substantially comprises the silicone gel.

In the embodiment where the net has been obtained by weaving, the meshes may be fixed by means of crossing threads so as to obtain a good dimensional stability.

In one embodiment of the invention, the yarn used for producing the net is preferably a continuous filament yarn, hardly extensible and non elastic, the extensibility preferably being lower than 35%.

Continuous filament yarn, means a yarn formed of one or several long downtwisted filaments. The choice of long filaments is to avoid short fibers which could detach from the net and become dispersed near the wound contact surface.

In one embodiment of the invention, the material of the yarns of the net is a synthetic material. Preferably, it consists of a hydrophobic material.

Thus, the material of the yarns of the net may be chosen from among polyesters (for example polyethylene terephthalates), polypropylenes, polyethylenes, polyam ides or even cellulose acetates. The advantage of these different materials is that they provide long filaments and yarns which have much less fibrils than the yarns obtained from short fibers.

The choice of some materials such as polyesters also allows thermosetting the open mesh structure of the net. Thus, according to one embodiment of the invention, the net is formed from continuous polyester yarns which have been thermo-set to form meshes. The advantages of such a net is that it is practically non extensible in the warp and weft directions and is worked more easily than other elastic nets. Such a structure of net ensures an even coating of the yarns of the net by the mixture which, let it be reminded, substantially comprises silicone gel. Thus, when the yarns of the net are formed of polyesters, this has the advantage that the coating by the mixture is facilitated.

In a preferred manner, the yarns of the net are of polypropylene and/or polyethylene.

In one embodiment of the invention, the net with open meshes is produced with yarns constituted of a same material, for example one of the aforementioned materials. In another embodiment of the invention, the net with open meshes is produced with warp and weft yarns which are constituted of different materials.

In a preferred manner, the open meshes of the net are wide, preferably of regular size and have a general for example round, oval or even polygonal form (such as rectangular or square). In an advantageous manner, the meshes have openings ranging between 1 and 5 mm on the side. The dimension of the meshes is such that the unitary surface of the openings ranges between 0.5 and 10 mm², preferentially of 0.5 to 3 mm². For example, it may consist in meshes of which the opening corresponds to approximately 4 to 20 meshes per cm.

The opening rate of the net (namely the ratio of the open surface of the net to the total surface of said net) advantageously ranges between 50 and 90%.

Once the net is coated with the mixture containing at least the silicone gel and the gelling agent, the opening rate of the net preferentially ranges between 40% and 90%, more preferentially between 75% and 90%.

In one embodiment of the invention, the water vapor transmission rate of the interface according to the invention ranges between 50 and 90 g/m²/24 hours, preferably between 75 and 90 g/m²/24 hours.

In a particularly advantageous manner, the net has been, prior to coating, washed so as to remove any enzyme adding traces or residues of a prior treatment to which the net yarns might have been subjected.

The net of the interface has been coated with a mixture substantially comprising silicone gel.

The silicone gel, a hydrophobic compound, stable in humid environment, allows imprisoning the net yarns which remain perfectly isolated from the wound as long as the interface according to the invention stays in place on the skin. Thus, there is no risk of direct contact between the yarns of the net and the regenerated tissue, which could lead to an inclusion of the yarns of the net into the wound, with as a result a painful destruction of the tissue upon removal of the interface.

In a preferred manner, the silicone gel is a mixture of a polydiorganosiloxane resin and catalyst, for example in a weight ratio of these aforementioned compounds of 1:1, 3:1 or even of 10:1, said polydiorganosiloxane and catalyst mixture having been subjected to cross-linking as it will be explained in further detail in the hereinafter description of the manufacture of the interface or compress according to the invention. The silicone gel used within the scope of the present invention may be that described in patent U.S. Pat. No. 4,991,574.

Preferably, the silicone gel is chosen from among the silicone gels which are perfectly compatible with the skin, preferably with low viscosities and pot life higher than 30 minutes.

Preferentially, the silicone gel is chosen from among the products of the:

WACKER company which are called SILPURAN, series 2110 and 2120,

BlueStar Silicones International company of series SILBIONE 4717 or even

NUSIL company which are known under CEREPLAST MED 634.

The cross-linking method of the silicone gel is perfectly within range of the one skilled in the art.

The mixture which coats the net of the interface further comprises at least one gelling agent.

The gelling agent has advantageously been dispersed evenly in the mixture of which the net of the interface according to the invention is coated, and thus preferentially in a relatively low quantity such that said mixture substantially comprising silicone gel (namely a hydrophobic compound) has a slightly hydrophilic nature, enough for maintaining a humid environment favorable to the aforementioned healing and which prevents a drying-out of the wound which could lead to an adherence of the interface, but insufficient to make the silicone gel capable of absorbing a lot of water. Indeed, this absorbent power of the silicone gel is not required as it would lead to a swelling of the mixture of which is coated the net of the interface; thus leading to a gradual obstruction of the meshes of the net.

Preferentially, the mass percentage of the gelling agent in the mixture of which the net is coated ranges between 0.1% and 20%, preferably between 5% and 15%, even more preferentially between 8% and 10%.

Higher quantities of gelling agent in the mixture could disrupt the cohesive nature of the silicone gel of the mixture; which would accentuate unacceptably the hydrophilic nature and modify considerably the slightly adhesive properties of the interface according to the invention thanks to the silicone gel.

In an advantageous manner, the gelling agent is chosen from among:

celluloses and their derivatives such as hydroxypropyl methyl cellulose, hydroxypropyl ethyl cellulose, methylcellulose, carboxymethyl cellulose and hydroxyethylcellulose, ethyl cellulose, ethylhydroxyethylcellulose, hydroxypropyl cellulose,

alginates such as sodium alginate,

alginic acid,

polyacrylic acid,

natural gums such as xanthan gum, tragacanth or even guar,

polyvinyl alcohol, polyvinylpyrrolidones and their derivatives,

copolymers of polyoxyethylenes and polyoxypropylenes,

chitosan,

pectins.

In a totally preferred manner, the gelling agent is carboxymethyl cellulose (abbreviated hereinafter by “CMC”). In fact, the advantage of CMC is that it can absorb up to 10 times its weight.

In one embodiment of the invention, the mixture in weight comprises from 5% to 15% CMC: thus allowing obtaining a mixture, which, after the coating thereof on a net, has a surface roughness remaining slightly adhesive and absorbent in contact with a wound.

Preferably, the CMC comes in the form of particles. The CMC particles are maintained dispersed in the mixture thanks to the cohesion of the silicone gel which allows their hydration and transformation into gel. When transforming into a gel, the CMC fibers hydrate the neighboring fibers which, in turn, are transformed into a gel and so on and so forth.

In one embodiment of the invention, the gelling agent comes in the form of a fine powder. Advantageously, the gelling agent is in solid form and finely divided, for example in the form of a powder of which the average granulometry may be less than 100 μm, preferably less than 50 μm. The silicone gel allows a homogenous dispersion of the gelling agent particles.

According to one embodiment of the invention, the gelling agent, preferably in the form of a fine powder, has been added to the silicone gel in a mixer-blender which has been subjected to a kneading, such that the dispersion thereof is homogenous in the silicone gel. The intimate mixing of the silicone gel and the gelling agent allows obtaining a mixture capable of maintaining a humid environment favorable to healing as has been detailed hereinabove.

In one embodiment of the invention, the mixture of which is coated the net of the interface further comprises at least one active principle, for example:

an active principle having a therapeutic effect intended for improving the healing properties of the interface, for example salicylic acid or hyaluronic acid,

an active principle having antiseptic properties, for example silver sulfadiazine, benzalkonium chloride or silver salt,

antibiotics such as for example neomycin or polymyxin,

anti-inflammatory agents such as for example triamcinolone acetonide.

Advantageously, the mixture comprises in weight between 1% and 10%, preferably between 1% and 5%, active principle.

In one preferred embodiment of the invention, the mixture comprises between 1% and 5% in weight of hyaluronic acid.

The choice and quantity of the active principles which are incorporated in the mixture which substantially comprises silicone gel are perfectly within range of the one skilled in the art.

The net of the interface according to the invention has been coated with a mixture which substantially comprises silicone gel. The coating has been achieved in such a manner as to leave most of the net meshes non obstructed while coating (or in other words called imprisoning) the yarns of the net with the mixture.

The quantity of mixture of which the yarns of the net are coated will depend on the structure of the net, namely the opening of the meshes thereof.

In one embodiment of the invention, the quantity of the mixture which substantially comprises silicone gel ranges between about 50 and 300 g/m², preferably between about 100 and 300 g/m², more preferentially between about 120 and 200 g/m², of the interface.

The interface according to the invention may be manufactured in the following manner.

As has been explained above, the silicone gel is a mixture of polydiorganosiloxane resin and a catalyst which has been subjected to cross-linking. linking.

More particularly, this polydiorganosiloxane resin and catalyst which is subjected to cross-linking results from the mixture of a 1^{st} composition comprising said polydiorganosiloxane resin and the catalyst in determined quantities with a 2^{nd} composition comprising said polydiorganosiloxane resin into a determined quantity and which is devoid of catalyst.

The cross-linking of the silicone gel (namely the polymerizing of the silicone io gel) is initiated as soon as the 1^{st} composition is mixed with the 2^{nd} composition (in other words, as soon as the 1^{st} composition is put into contact with the 2^{nd} composition), and thus as a result of the choice of determined quantities of the constituents of the 1^{st} and 2^{nd} compositions for the cross-linking to start.

The choice and quantities of the constituents of the 1^{st} and 2^{nd} compositions with a view to obtaining a cross-linked silicone gel are perfectly in the range of the one skilled in the art. As soon as the 1^{st} and 2^{nd} compositions are combined, the cross-linking is initiated. Then, a cross-linking step of which the conditions are detailed hereinafter is implemented.

The method of manufacturing the interface according to the invention may comprise the following steps:

a) a 1^{st} preparatory mixture comprising at least one gelling agent is prepared and possibly at least one active principle such as described above. Preferably, the gelling agent and possible active principles come in the form of a powder.

b) Then, this 1^{st} preparatory mixture is incorporated in a first composition which comprises at least one polydiorganosiloxane resin and a catalyst in determined quantities. The incorporation of this 1^{st} preparatory mixture is carried out in a regular manner, for example during a time period of 1 minute, in a mixer-blender operating at low speed. In one embodiment, the mixer-blender is a planetary type mixer-blender and the rotation of the blade ranges between 50 and 100 revolutions/minute. Following this incorporation of this 1^{st} preparatory mixture in the 1^{st} composition, a 2^{nd} preparatory mixture is obtained.

c) Then, in this 2^{nd} preparatory mixture thus obtained is incorporated a 2^{nd} composition which comprises at least a determined quantity of polydiorganosiloxane resin and which is devoid of catalyst.

d) The whole mixture thus obtained at step c) is mixed at low speed, for example with a rotation of the blade ranging between 50 and 100 revolutions/minute, and thus until obtaining a homogenous mixture which substantially comprises silicone gel.

e) The mixture obtained at step d) is coated on the yarns of a net.

f) The silicone gel comprised in the mixture is cross-linked.

Steps a) to d) described above may be carried out both continuously and discontinuously (in other words called “batch mode”).

Due to the aforementioned mixing of the 1^{st} composition and the 2^{nd} composition, the cross-linking of the silicone gel is initiated during this step c) and a mixture is obtained at step d), a mixture which substantially comprises silicone gel.

In an advantageous manner, during step c) of incorporating in the 2^{nd} preparatory mixture the 2^{nd} composition, a degassing of the air bubbles contained in this mixture is carried out so as to improve the cohesion of the silicone gel after cross-linking. In one embodiment of the invention, said degassing is carried out by putting the mixture under depression, for example between 0.5 and 0.1 bar.

The mixture thus obtained at step d) has a high viscosity, preferably ranging between 10 000 and 100 000 cps. This is why the method of coating the net is suitable to take into account this high viscosity of said mixture.

Advantageously, the net comes in the form of a strip.

The coating of the mixture on the net may be carried out according to the steps such as detailed below, and thus for example in a coating facility. Preferably, the coating facility has suitable elements which are perfectly within the range of the one skilled in the art for coating the net which comes in the form of a strip.

First, the net of the interface is entirely soaked in said mixture of which the preparation has been described above.

The net is thus charged with an excess of said mixture. The net is progressively laminated to then be taken under a first coating doctor blade which pre-regulates the spread of the mixture disposed on the net. Then, said net is taken under a second coating doctor blade for finely regulating the final spread of the mixture deposited on said net.

In another embodiment of the invention, the net is passed into a bath comprising the mixture such as described hereinabove. The net covered with said mixture is then passed between two rotary cylinders pressed against each other with a predetermined spacing such as to eliminate the excess from the mixture. The quantity of mixture remaining on the yarns of the net substantially depends on the spacing imposed between the stationary cylinders.

So as to ensure optimal permeability of the net after the coating, a blowing is carried out on the net thus coated with the mixture, only at the yarns, for example in a vertical ventilation area so that the mixture does not run and remains regularly coated around the yarns and the airing of the grid is perfectly mastered.

A jet of air can also be applied on the net after passing under the coating doctor blade or between two rotary cylinders pressed against each other.

In one embodiment of the invention, after the coating step e), the regular airing of the net so that the meshes of the net remain open after the coating is obtained by air blowing and/or the passage of the net on a stainless steel cylinder such that the excess mixture remains bonded on the stainless steel cylinder. Preferentially, this stainless steel cylinder is then scraped such as to continuously clean it and recycle excess mixture.

In one embodiment of the invention, a laminar air jet system may be integrated in the coating facility for correcting the distribution of the mixture around the yarns and unblocking the mesh openings which would not have been opened during the passage in the second cylinder.

With this aforementioned coating method, the spreads of said mixture coated on the net advantageously range between 100 g/m² and 300 g/m². This corresponds to a coating thickness of said mixture ranging between 200 and 600 μm. Thus, the assembly of the meshes of the net is coated with the mixture and approximately 50% of the available surface is coated with the mixture, the other 50% of the surface remain perfectly open and permeable to the exudates of the wound and to air.

According to another embodiment of the invention to be considered, the silicone gel possibly comprising active principles has been coated on the net such as to coat the yarns and leave the mesh openings non obstructed, for example such as has been detailed hereinabove and, while the silicone gel has not yet been cross-linked, fine particles of gelling agent are projected onto the surface of the silicone gel. In this embodiment of the invention, the quantity of gelling agent may be less than that used when the gelling agent has been incorporated in the silicone gel before coating on the net.

Moreover, this embodiment of the invention allows obtaining an interface of which the face intended to be exposed in direct contact of the wound has received by projections a gelling agent such that only this face of the interface has a hydrophilic nature, the rest of the interface remaining hydrophobic.

This is why, according to this variant of the invention, the weight of gelling agent may represent approximately between 0.1% and 1% of the weight of the mixture. This quantity will be sufficient for the interface according to the invention to have an amphiphilic nature on the surface which is satisfactory for creating favorable humidity for the aforementioned wound healing.

Then, the net coated with the mixture is subjected to a cross-linking step such that the silicone gel substantially comprised in the mixture cross-links. Thereby an interface is obtained according to the present invention.

The parameters of the cross-linking of the silicone gel are perfectly within the range of the one skilled in the art.

The cross-linking is advantageously carried out according to a gradient of suitable temperatures in a furnace. The furnace in which the cross-linking step is carried out is perfectly within range of the one skilled in the art.

For example, a temperature gradient may be the one detailed below in table 1:

TABLE 1
temperature gradient during the cross-linking step
of the mixture with which the net is coated

| | Time (in seconds) | | | | | |
|---|---|---|---|---|---|---|
| | 0 | 15 | 30 | 60 | 90 | 120 |
| Cross-linking | 20 | 40-60 | 80-90 | 120-130 | 130-140 | 140 |
| temperature (° C.) | | | | | | |

In one embodiment of the invention, the cross-linking step of the mixture which substantially comprises silicone gel is carried out in approximately 2 minutes at a temperature of 120° C.

It is worth noting that when the interface is intended to cover an absorbent material as is described hereinafter, it is also considered within the scope of the present invention to apply the net coated with the mixture on the absorbent material, then to subject the assembly thus constituted by the interface and the absorbent material to a cross-linking step, and thus in the same manner as has been described hereinabove.

In this manner, a resistant bond is obtained between the net coated with the mixture and the absorbent material. In fact, in this embodiment of the invention, the mixture which substantially comprises silicone gel will penetrate in the absorbent material and be absorbed by the latter before the cross-linking of the silicone gel is completed. In this manner, the interface thus obtained will be fixed to the absorbent material in a more cohesive manner than if it had been fixed to the absorbent material after completing the cross-linking of the silicone gel.

When the net comes in the form of a strip, following the cross-linking, it is cut into individual interfaces according to dimensions suited to the use of the interface. The interface may be packed into sealed and sterilized sachets.

Another object of the present invention is a compress which comprises the interface such as described hereinabove.

In one embodiment of the invention, said compress comprises at least one interface such as described hereinabover and at least one absorbent material, said interface being fixed on the absorbent material. Thus, the assembly constituted by the interface and the absorbent material forms the compress.

In one embodiment the absorbent material may be chosen from among:

polyurethane foams, and more preferentially aliphatic polyurethane foams,

non-woven fabric such as viscose non woven fabric or polyester and viscose based mixture (for example, a mixture comprising in weight 70% to 80% viscose and 20 to 30%polyester), or even the non-woven fabric containing cellulose,

cellulose materials,

the non woven compresses formed from fibers, for example superabsorbent fibers such as polyacrylate fibers.

The choice of absorbent material is perfectly within the range of the skilled person.

The advantage of the aliphatic polyurethane foam is that it is a flexible absorbent foam with open cells allowing for an excellent retention level by about 65%. It also has absorption capacities of more than 15 times its weight. Its thickness may vary from 1.5 to 5 mm. In addition, it has the advantage of resisting to the irradiation of beta and gamma rays. This is particularly suitable when the compress according to the invention can be sterilized, thereby implying that all its constitutents are able to be sterilized.

Thus, during exudation of the wound, the absorbent materials absorb the exudates through the meshes of the net which has been coated with a mixture substantially comprising silicone gel.

As it has been explained hereinabove, the compress according to the invention may be manufactured via fixing by bonding an interface according to the invention on an absorbent material. In other words, it is fixed on an absorbent material a net with open meshes which has been coated with a mixture comprising at least a silicone gel and at least a gelling agent. The silicone gel is in cross-linked form

In another embodiment of the invention, the compress according to the invention is manufactured by disposing a net with open meshes which has just been coated with said mixture comprising at least a silicone gel and at least a gelling agent on an absorbent material, then by subjecting the assembly comprisig the net coated with the mixture and the absorbent material to a cross-linking step of the silicone gel. It is thus obtained a more cohesive compress than according to the other mode of manufacturing of the compress according to the invention wherein the interface according to the invention is fixed (for example by bonding) on an absorbent material after cross-linking the silicone gel.

Another object of the invention is a dressing including at least one compress according to the invention which is bonded on a support, said support has at least one adhesive area configured for fixing said dressing on a skin, and in particular at the peripheral areas of a wound.

In a preferred manner, the dressing according to the invention can be sterilized. This means that all its constituents can be sterilized. This sterilization property is essential if the dressing according to the invention is intended to be applied on acute, chronic and exudate wounds. These wounds are often found on weakened people (for example people with severe burns, people at the end of life). It is thus essential to sterilize the dressing so as to prevent contamination risks.

In one embodiment of the dressing according to the invention, a layer of an absorbent material is intercalated between the absorbent material of the compress and the support which includes the adhesive area. This gives the advantage of reinforcing the capacity of retention of the exudates of the dressing according to the invention.

In a preferred manner, the compress is centered on the support of the dressing.

In one embodiment of the dressing according to the invention, the compress is disposed in a centered manner on a hypoallergenic support which includes at least one adhesive area which is a layer of acrylic glue or silicone gel, and thus such as to maintain the dressing in place on the patient according to the invention.

When the adhesive area is a layer of silicone gel, it allows an atraumatic removal of the dressing according to the invention.

According to one embodiment of the dressing, the support comprises a polyurethane film which is covered with a non woven layer, the non woven layer being itself covered with a layer of acrylic glue or silicone gel. The absorbent material of the compress is fixed on this layer of acrylic glue or silicone gel, preferably in a centered manner. The absorbent material is covered with the interface according to the invention. For example, the interface according to the invention has been fixed (in particular by bonding) on said absorbent material. According to a variant of this embodiment of the invention, a layer of super-absorbent material has been intercalated between the layer of acrylic glue or silicone gel and the absorbent material.

The structure of the support described hereinabove has the advantage of correctly maintaining in place the interface according to the invention on the wound, and thus even when the patient is taking a shower. In fact, the polyurethane film in the structure of the support such as described hereinabove is advantageous due to the fact that it enables the dressing to be imperviousness to water while keeping a very good permeability to water vapor and an excellent conformability of the dressing according to the invention.

Furthermore, with such a support structure, the dressing according to the invention may easily be put in place on the wound needing to be healed by the staff or the patient himself/herself.

In the embodiment of the dressing according to the invention also to be considered wherein the support is devoid of polyurethane film and comprises, for example, a layer of non woven fabric covered with an adhesive layer, this dressing will not have a good enough hold in the shower as the aforementioned dressing with a polyurethane film in the support structure.

In another embodiment of the invention, the dressing according to the invention comprises at least a layer of material which is impregnated with antiseptic active principles such as chlorhexidine and/or silver salt.

In one embodiment of the invention, the absorbent material of the compress has been impregnated with these antiseptic active principles.

In another embodiment of the invention, these antiseptic active principles have been incorporated in the mixture which substantially comprises silicone gel described above.

In one embodiment of the invention, the adhesive property of the adhesive area consists in acrylic glue or a silicone gel which allows removing the dressing atraumatically.

The dressing according to the invention is particularly advantageous when the compress such as described hereinabove is intended to be applied on exudative or highly exudative wounds.

Indeed, the absorbent material will absorb the wound exudates as they appear.

The interface comprised by the dressing according to the invention is:

non adherent due to the fact that the net has been coated with a mixture which substantially comprises silicone gel,

permeable to wound exudates due to the fact that it comprises a net with open meshes non obstructed by the coating (that is to say, keeping an opening between each of the meshes), and thus by avoiding any direct contact of the wound with the absorbent material.

In one embodiment of the invention, the dressing further comprises at least one protective film which is arranged for covering the interface and the adhesive area.

The material of the protective film may be a polyester (for example a polyethylene terephthalate) coated with a fine layer of fluorosilicone or a polyethylene, preferably a film of crimped polyethylene which is compatible with the mixture of which is coated the net and with the adhesive of the adhesive area of the support. In one embodiment of the invention, the material of the protective film is a polyethylene terephthalate coated with a layer of fluorosilicone.

It is advantageous to use a polyethylene film, as this polymer by nature hardly adheres to the silicone gel. In a preferred manner, embossed and/or crimped films are used so as to reduce the contact surfaces and thus allow removing this film easily from the interface. The crimped studded polyethylene films of the RKW company, 55 μm and 65 μm are preferred.

Thus, the material of the protective film is suitably chosen for being non adherent with respect to the silicone gel of the interface.

In one embodiment of the invention, the protective film such as described above is deposited on the net of the interface coated with the mixture which substantially comprises silicone gel, and thus right after the aforementioned cross-linking step which is advantageously carried out in a furnace. For example, after coming out of the furnace in which the cross-linking step has been carried out, a film of crimped polyethylene is deposited on the net coated with mixture. The protective film thus slightly catches on the mixture of which the net is coated and is driven by the latter in the production line of the interface according to the invention. It is not necessary to apply pressure on the protective film.

According to one embodiment of the invention, the dressing further comprises a packaging in which it is placed prior to the use thereof, said packaging comprises for example two thermo-sealable films which can be sterilized by radiation or ethylene oxide or by any other means for sterilizing medical or pharmaceutical products within the range of the skilled person.

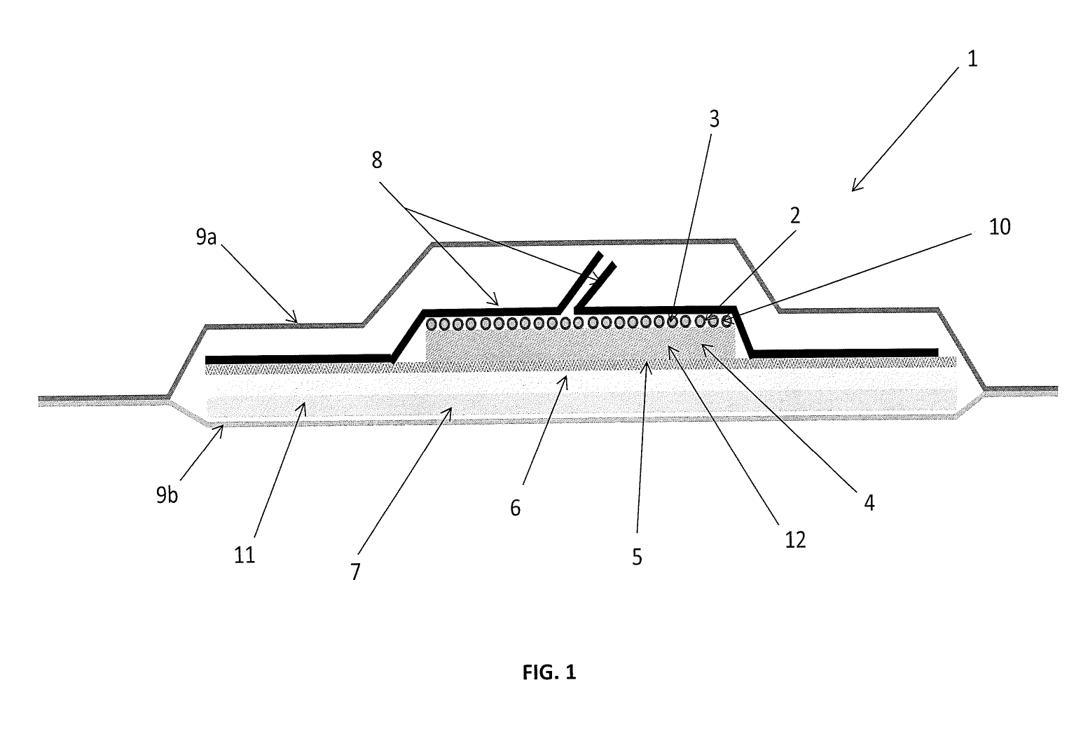

Other features and advantages of the invention will become apparent during the following description of the dressing according to the invention, given by way of non limiting example, with reference to the accompanying drawing wherein:
FIG. 1 is a schematic view according to a longitudinal section of a dressing according to the invention.

FIG. 1 represents a longitudinal section of a dressing 1 according to the invention. The dressing 1 comprises an interface 10. Said interface 10 comprises a polyester net 2 which has been coated with a mixture 3 which comprises 89% in weight of silicone gel, 1% hyaluronic acid and 10% CMC. The net covers an absorbent material 4 which is a polyurethane foam.

The assembly constituted by the interface 10 and the absorbent material 4 is a compress 12 according to the invention which is disposed in a centered manner on a support 11. More particularly, the support 11 comprises a polyurethane film 7 which is covered with a layer of non woven fabric 6. The layer of non woven fabric 6 is covered with a layer of acrylic glue 5 which is hypoallergenic.

Two protective films 8 of crimped polyethylene cover the compress 12, as well as the area of acrylic glue layer 5 which is not covered by the compress 12.

The dressing 1 is sealingly packed in a sealed package which includes:

a first transparent face 9a in a material comprising a mixture of polyester, polyethylene and polyethylene terephthalate (hereinafter called “PET”), and

a second transparent face 9b in a material comprising a mixture of aluminized PET, metallic polyester and polyethylene on which the polyurethane 7 film rests

### EXPERIMENTAL SECTION

The decrease in the adherence of an interface according to the invention based on the formation of the gel has been tested.

To this end, the peel force at 180° has been measured on a steel sheet of an interface according to the invention, and thus according to the hydration thereof.

More particularly, the interface used during these tests measured 4 cm wide and 10 cm long and comprised a polyethylene net which had been coated with a mixture comprising 90% in weight of silicone gel and 10% CMC, according to a spread of 200g/m².

The weight in water, expressed in grams, having been deposited on the interface is detailed in the table 2 below.

Each interface hydrated in this manner has been bonded onto the steel sheet.

Using a dynamometer, it has been measured according to standard NF EN 1943 the force required for detaching said hydrated interface from the steel sheet.

Furthermore, the formation of gel has been observed at the surface of the interface. The percentage of the surface of the interface on which has been observed the formation of gel is detailed in the table 2 hereunder:

TABLE 2
detailing the peel force and the percentage of gel formed
and observed at the surface of the interface according
to the weight of the water deposited on the interface

| | Weight of water deposited in g | | | |
|---|---|---|---|---|
| | 0 | 0.25 | 0.5 | 0.75 |
| Peel force measured | Between | Between | Between | 0 |
| in newtons (N) | 0.60 and | 0 and | 0 and | |
| | 0.80 | 0.80 | 0.60 | |
| Gel surface in % | 0 | 30% | 65% | 100% |

Based on the table 2, it is thus noted a decrease in the adhesive strength of the interface according to the invention with the formation of the gel which is also proportional to the absorbed water.

It is worth noting, that from absorption of 0.75 g water over 40 cm² of an interface according to the invention, it is no longer possible to measure any adhesive strength to the tangential detachment (in other words called peel force).

Between 0.25 g and 0.75 g of absorbed water over 40 cm² of an interface according to the invention, the adhesive strength (in other words the peel force) decreases and the measurements are very “chaotic”: there are areas which are perfectly non adherent (peel force zero) and a few points of resistance corresponding to areas which are not entirely hydrated where low adherences ranging between 0.60 and 0.80 N are measured.

In addition, absorption and retention tests have been undertaken respectively on:

a 1^{st} circular compress of 50 mm of diameter composed of, in percentages of weight with respect to the total weight of said compress, 25% viscose fibers, 25% polyester fibers and 50% polyacrylate fibers and which further included an interface according to the invention such as described hereinabove in the experimental section, namely a compress according to the invention, and

on a 2^{nd} compress having the same features as the 1^{st} compress but was devoid of an interface according to the invention, namely a comparative compress.

The tests have been carried out according to the experimental protocol described in chapter 3.3 called “fluid retention capacity” of standard NF EN 13726-1.

The solution used was a sodium chloride solution comprising 8.66 g and completed to a liter of water. The temperature of the climatic enclosure was 37° C. The measurements were carried out after 24 to 48 hours.

Table 3 herebelow details:

the mass of fluid absorbed on the compress tested after 24 hours and 48 hours,

specific retention of the fluid on the compress tested after 24 hours and 48 hours.

TABLE 3
detailing the mass of absorbed fluid and the fluid
retention capacity after 24 and 48 hours.

| | Mass of | Fluid | Mass of | Fluid |
|---|---|---|---|---|
| | absorbed | retention | absorbed | retention |
| | fluid after | capacity after | fluid après | capacity after |
| | 24 h | 24 h | 48 h | 48 h |
| | (g/24 h) | (9/24 h) | (g/48 h) | (g/48 h) |
| Compress | 1.9848 | 3.2284 | 2.0437 | 4.1222 |
| according to | | | | |
| the invention | | | | |
| Comparative | 2.0134 | 3.3024 | 2.0909 | 4.2146 |
| compress | | | | |
| Variations in | −1.4% | −2.2% | −2.2% | −2.2% |
| performance | | | | |

The differences between the absorption and retention capacities of the compress according to the invention and the comparative compress only vary by 1.4% to 2.2%. This is not significant in light of the risks of these measurements.

Thus, an interface according to the invention remains perfectly open during the use thereof and may leave the wound exudates pass through without any obstacle thanks to its excellent opening and permeability. In other words, there is no obstruction of the open meshes of the net of the interface according to the invention during the use of the compress according to the invention.

1. A non adherent healing interface including at least one net with open meshes, wherein the net has been coated with a mixture comprising at least silicone gel and at least a gelling agent.

2. The interface according to claim 1, wherein at least part of said meshes are not obstructed by said mixture comprising at least silicone gel and at least a gelling agent.

3. The interface according to claim 1, wherein once said net is coated with the mixture containg at least silicone gel and the gelling agent, the opening rate of the net ranges between 40% and 90%.

4. The interface according to claim 1, wherein the water vapor transmission rate of the interface ranges between 50 and 90 g/m²/24 hours.

5. The non adherent healing interface according to claim 1, wherein the weight in silicone gel represents at least 80% of the total weight of said mixture.

6. The non adherent healing interface according to claim 1, wherein the material of the yarns of the net is chosen from among polyesters, polypropylenes, polyethylenes, polyamides and cellulose acetates.

7. The non adherent healing interface according to claim 1, wherein the mass percentage of the gelling agent in said mixture ranges between 0.1% and 20%.

8. The non adherent healing interface according to claim 1, wherein the gelling agent is chosen from among hydroxypropyl methyl cellulose, hydroxypropyl ethyl cellulose, methylcellulose, carboxymethyl cellulose, hydroxyethylcellulose, ethyl cellulose, ethylhydroxyethylcellulose, hydroxypropyl cellulose, alginates such as sodium alginate, alginic acid, polyacrylic acid, xanthan gum, tragacanth gum, guar gum, polyvinyl alcohol, polyvinylpyrrolidones, copolymers of polyoxyethylenes and polyoxypropylenes, chitosan, pectins.

9. The non adherent healing interface according to claim 1, wherein the quantity of the mixture ranges between 50 and 300 g/m² of said interface.

10. A method of manufacturing an interface according to claim 1, wherein it comprises at least the following steps:
a) a 1^{st} preparatory mixture comprising at least one gelling agent is prepared;
b) This 1^{st} preparatory mixture is incorporated in a 1^{st} composition which comprises at least one polydiorganosiloxane resin and a catalyst so as to obtain a 2^{nd} preparatory mixture;
c) In this 2^{nd} preparatory mixture thus obtained is incorporated a 2^{nd} composition which comprises at least the polydiorganosiloxane resin and is devoid of catalyst;
d) The whole mixture thus obtained at step c) is mixed until obtaining a homogenous mixture which substantially comprises silicone gel;
e) The mixture obtained at step d) is coated on the yarns of a net;
f) The silicone gel comprised in said mixture is cross-linked.

11. The method of manufacturing an interface according to claim 10, wherein step e) of coating the mixture on the yarns of the net comprises at least steps:
a) The net is entirely soaked in said mixture such that the net is charged with an excess of said mixture;
b) The net is laminated;
c) The net is taken under a 1st coating doctor blade which pre-regulates the spread of the mixture disposed on the net;
d) The net is taken under a 2^{nd} coating doctor blade for finely regulating the final spread of the mixture deposited on said net.

12. The method of manufacturing an interface according to claim 10, wherein after the coating step e), a blowing of air is carried out on the net and/or the net is passed on a stainless steel cylinder which is configured so that the excess mixture remains bonded on said stainless steel cylinder.

13. A compress comprising at least one interface according to claim 1 and at least one absorbent material, said interface being fixed on said absorbent material.

14. A method of manufacturing a compress according to claim 13, wherein a net which has just been coated with the mixture is placed on an absorbent material, then the whole comprising the net coated with the mixture and the absorbent material is subjected to a step of cross-linking of the silicone gel.

15. A dressing including at least one compress according to claim 13 which is bonded on a support, said support having at least an adhesive area configured for fixing said dressing on the skin.
